(19) United States
(12) Patent Application Publication
(10) Pub. No.: US 2023/0129864 A1
(43) Pub. Date: Apr. 27, 2023

(54) METHOD AND APPARATUS FOR STERILIZING MEDICAL INSTRUMENTS

(71) Applicant: LOG10 B.V., Eindhoven (NL)

(72) Inventors: Franciscus Maria VERHOEVEN, Amsterdam (NL); Theo Alex Eduard VAN DER LEIJ, Moergestel (NL); Mirte PEETERS, Dongen (NL); Thijs DE JONG, De Bilt (NL)

(21) Appl. No.: 17/910,636

(22) PCT Filed: Mar. 12, 2021
(86) PCT No.: PCT/NL2021/050170

§ 371 Date: Sep. 9, 2022

(30) Foreign Application Priority Data
Mar. 12, 2020 (NL) 2025110

(51) Int. Cl. A61L 2/14; A61L 2/26; F24F 6/00

(52) U.S. Cl. CPC A61L 2/14; A61L 2/26; F24F 6/00; A61L 2202/14; A61L 2202/122; A61L 2202/123; A61L 2202/24

(57) ABSTRACT

A method and apparatus for sterilizing medical instruments, including a device for controlling specific humidity, SH, of flow of a gas or gas mixture. The device includes a tank for storing a volume of water. The tank has an inflow port for receiving the gas or gas mixture, and an outflow port for allowing humidified gas or gas mixture at the required SH to exit the tank. The temperature of the water and/or the pressure of the supplied gas or gas mixture are controlled for the required SH.

### FIELD OF THE INVENTION

The present invention relates to sterilizing medical instruments, such as dental instruments. More specifically the invention relates to sterilizing using a plasma source. Even more specifically the invention relates to providing a gas or gas mixture, such as air, having a well-defined specific humidity to the plasma source.

### BACKGROUND TO THE INVENTION

Reusable medical instruments are instruments that health care providers can reuse to diagnose and/or treat multiple patients. Examples of reusable medical instruments include medical instruments used in dental care, such as scalpels, syringes, scopes, mirrors, drills, burs, discs, handpieces, excavators, turbines, files, reamers, etc.

When used on patients, reusable instruments become soiled and contaminated with blood, tissue and other biological debris such as microorganisms. To avoid any risk of infection by a contaminated instrument, the reusable instruments can be sterilized. Sterilizing results in a medical instrument that can be safely used more than once in the same patient, or in more than one patient. Adequate sterilizing of reusable medical instruments is vital to protecting patient safety.

Various sterilizing agents can be used for sterilizing medical instruments. Historically, steam or hydrogen peroxide is often used. More recently, plasma devices are being used for ionizing gases or gas mixtures, the ionized gas being used as sterilizing agent. Electrons in the plasma impact on gas molecules causing dissociation and ionization of these molecules, which creates a mix of reactive species. It is known to directly expose the medical instruments to the plasma, or to expose the medical instruments to the (partially) recombined plasma, sometimes referred to as afterglow, see e.g. S. Moreau et al., “Using the flowing afterglow of a plasma to inactivate Bacillus subtilis spores: Influence of the operating conditions”, J. Appl. Phys. Vol. 88, No. 2, 15 Jul. 2000.

Several attempts have been made to improve upon plasma sterilizing. US2011/0027125A1 discloses a system comprising a chamber and a plasma generator for generating free radicals combined with use of a hydrogen peroxide solution.

In sterilizing, plasma sources often use a gas or gas mixture, such as air, flowing through the plasma source to be ionized. The moisture content of the gas flow, or specific humidity, can be important in determining the reactive components being generated in the plasma source. However, proper control of the specific humidity of the gas stream tends to be difficult and expensive.

There exists a need for more efficient and effective plasma sterilizing.

### SUMMARY OF THE INVENTION

It is an object to provide a method and system for sterilizing medical instruments, such as dental instruments. It is an object to provide a more efficient and/or effective method and system for sterilizing medical instruments, such as dental instruments. It is also an object to provide a device and method for controlling specific humidity of a gas stream, particularly for being fed into a plasma source, especially for sterilizing medical instruments.

Thereto, according to an aspect is provided an apparatus for sterilizing medical instruments, such as dental instruments. The sterilizing apparatus includes a device for controlling specific humidity, SH, of flow of a gas or gas mixture, such as air. It has been found that controlling specific humidity of a gas stream fed to an instrument to be sterilized is beneficial for the sterilizing process. Especially when using a plasma sources the specific humidity of the gas flow being ionized by the plasma source can be important in determining the reactive components being generated in the plasma source. The device for controlling specific humidity includes a tank for storing a volume of water. The tank has an inflow port for receiving the gas or gas mixture. The temperature of the water and/or the pressure of the supplied gas, or gas mixture, are adjusted for the required SH. The tank has an outflow port for allowing humidified gas or gas mixture at the required SH to exit the tank.

Specific humidity, SH, or moisture content, is the ratio of the mass of water vapor to the total mass of the humid gas. Specific humidity is a useful parameter, as it relates to the actual amount, i.e. mass, of water vapor present in a given amount of gas. The specific humidity does not vary as the temperature or pressure of a body of air changes, as long as moisture is not added to or taken away from it. Specific humidity, however is difficult to measure. Also it is difficult to set a desired specific humidity. Certain techniques of setting specific humidity rely on in a first step completely removing all moisture from a gas, followed by in a second step evaporating a known amount of moisture into the gas. This can be quite cumbersome and expensive.

Relative humidity, RH, is the ratio of the partial pressure of water vapor to the equilibrium vapor pressure of water at a given temperature. Relative humidity depends on temperature and the pressure of the system of interest. The same amount of water vapor results in higher relative humidity in cool air than warm air. Relative humidity is easier to measure than specific humidity.

The relationship between specific humidity and relative humidity is strongly dependent on temperature and pressure. It has been found that specific humidity can be controlled by controlling temperature, relative humidity and pressure. In particular, when relative humidity is fixed, specific humidity can be controlled by controlling temperature and pressure. Relative humidity, RH, can be fixed at 100% if the gas, or gas mixture, is saturated with water vapor. By providing the tank storing a volume of water and having an inflow port for receiving the gas or gas mixture, the gas or gas mixture can easily be maintained at a relative humidity of 100% inside the tank. The inflow port can be arranged to have the gas or gas mixture enter the volume of water below the water surface. Hence, the having the gas or gas mixture pass through the water aids in having the RH at 100%. The outflow port can be in communication with a gas head inside the tank

The relationship between pressure, temperature and SH for a saturated humid gas or gas mixture is given by the following equation 1.

$${P}_{tot} ( T , SH ) = \frac{A \cdot {10}^{\frac{m \cdot T}{T + {T}_{n}}}}{1000} \cdot ( 1 + \frac{{M}_{H 2 O}}{M dry} \cdot ( \frac{1000}{SH} - 1 ) ) \qquad EQ 1$$

Herein Pₜₒₜ is the total pressure of the humid gas or gas mixture (in bar(a)), T is the temperature of the humid gas or gas mixture (in ° C.), SH is the specific humidity of the humid gas or gas mixture (in grams of water vapor per kilogram of gas), M_{dry} is the molar mass of the dry gas or gas mixture (in g/mol), and M_{H2O} is the molar mass of water (in g/mol). The parameters A, in and Tₙ are constants. The below table gives values for these constants for water, for several temperature ranges.

| | | | | Temperature | |
|---|---|---|---|---|---|
| A | m | Tₙ | max error | range | |
| 6.116441 | 7.591386 | 240.7263 | 0.083% | −20 . . . +50° | C. |
| 6.004918 | 7.337936 | 229.3975 | 0.017% | +50 . . . +100° | C. |
| 5.856548 | 7.27731 | 225.1033 | 0.003% | +100 . . . +150° | C. |
| 6.002859 | 7.290361 | 227.1704 | 0.007% | +150 . . . +200° | C. |

In a practical example, e.g. for water at 40° C., A=6.116441, m=7.591386, Tₙ=240.7263, M_{dry}=28.9444 for air g/mol, and M_{H2O}=18.01534 g/mol. From this relationship the required pressure can be calculated for a desired SH at a given temperature. Similarly, from this relationship the required temperature can be calculated for a desired SH at a given pressure.

As stated, when relative humidity is fixed specific humidity can be controlled by controlling temperature and pressure. Therefore, using this relationship there is no need to first remove all moisture from a gas and subsequently add a known amount of moisture. Instead is relied on the known saturation of the gas with water vapor and its relation with the controllable temperature and/or pressure. It will be appreciated that it is possible that temperature is maintained and pressure is adjusted for the desired SH. It is also possible that pressure is maintained and temperature is adjusted for the desired SH. It is also possible that both temperature and pressure are adjusted for the desired SH.

Optionally, the apparatus includes a processor arranged for receiving a desired SH value as input and for determining suitable values for temperature and/or pressure, e.g. on the basis of equation EQ1. The processor can be arranged for receiving or determining the desired SH value and for determining values for a required temperature and/or required pressure on the basis of the desired SH value, e.g. on the basis of equation EQ1.

Optionally, the apparatus includes a temperature sensor and temperature controller for maintaining the water at the controlled temperature. When the water temperature is maintained at a controlled value, variations in SH can be minimized. The processor can be arranged for receiving a desired SH value and the controlled temperature value as input, and for determining a required pressure, on the basis of the desired SH and the controlled temperature, e.g. on the basis of equation EQ1.

Optionally, the apparatus includes a pressure regulator, e.g. in communication with the inflow port. Hence, the pressure of the gas or gas mixture in the tank can be controlled and maintained. The processor can be arranged controlling the pressure to be the required pressure as determined on the basis of the desired SH and the controlled temperature, e.g. on the basis of equation EQ1. When the gas pressure inside the tank is maintained at a controlled value, variations in SH can be minimized.

It will be appreciated that it is possible that the processor is arranged for receiving a desired SH value the controlled pressure value as input, and for determining a required temperature, on the basis of the desired SH and the controlled pressure, e.g. on the basis of equation EQ1. The processor can then be arranged for controlling the temperature to be the required temperature as determined on the basis of the desired SH and the controlled pressure, e.g. on the basis of equation EQ1.

Optionally, the apparatus includes a water supply port for maintaining the water level within a predetermined interval. Preferably, the water supply port includes a level meter for determining the water level in the tank. Thus the water level can automatically be maintained.

Optionally the apparatus includes a plasma source having an ionization chamber in communication with the device for controlling specific humidity. Optionally, the outflow port of the device is connected to a gas input of the ionization chamber via a choke. Thus, the gas or gas mixture having the controlled SH can be fed to the ionization chamber of the plasma source. The controlled SH can help in setting or maintaining the reactive components being generated in the plasma source.

It will be appreciated that the processor can be arranged for receiving an indication representative of desired sterilization parameter and determining a desired SH value on the basis thereof. Such sterilization parameter can e.g. be a desired reactive component content of the plasma. The reactive component content of the plasma can be dependent on the SH of the gas or gas mixture fed to the ionization chamber of the plasma source. The processor can e.g. look up a desired SH value corresponding to the desired sterilization parameter, such as the desired reactive component content, in a database or memory. It will be appreciated that the indication representative of the desired sterilization parameter can e.g. be one of a set of predetermined settings of the sterilization apparatus. The predetermined settings can e.g. relate to different instruments to be sterilized, different grades of contamination of the instruments to be sterilized, different levels of sterilization, or the like.

Optionally, the outflow port of the device is connected to a gas input of the ionization chamber via a choke. Thus, pressure inside the tank can be maintained while the gas flow can be fed to the ionization chamber at a reduced pressure relative to the pressure inside the tank.

Optionally, the apparatus includes one or more of a RH sensor, temperature sensor, pressure sensor, and flow sensor in a connection from the outflow port of the device to a gas input of the ionization chamber. These sensors can be used for verifying the RH, temperature, pressure, and/or flow of gas input to the ionization chamber.

Optionally, the apparatus includes a chamber arranged for placing the medical instrument therein. The apparatus can include a temperature control unit arranged for controlling the temperature of the medical instrument and/or the chamber such that the temperature of the medical instrument is below the temperature of chamber for allowing the sterilizing agent to at least partially condense onto the instrument.

Optionally, the apparatus further includes a washing unit arranged for washing and/or rinsing the medical instruments prior to sterilization. A cooling gas stream, e.g. including atomized water, can be supplied to the washed medical instruments for drying and cooling the medical instruments.

It will be appreciated that the device for controlling specific humidity of flow of a gas or gas mixture, including a tank for storing a volume of water, wherein the tank has an inflow port for receiving the gas or gas mixture, wherein the temperature of the water and/or the pressure of the supplied gas or gas mixture are controlled for the required SH, and wherein the tank has an outflow port for allowing humidified gas or gas mixture at the required SH to exit the tank, can also be used for supplying a flow of gas having controlled SH in different applications than in a sterilization apparatus for sterilizing medical instruments. Such device can e.g. be used in combination with a plasma source. It will be appreciated that a plasma source can be provided having an ionization chamber, one or more electrodes, and an electric power source. The ionization chamber can have an input for feeding humidified gas, such as humidified air, into the ionization chamber. The plasma source can include a humidifier providing the humidified gas to the ionization chamber. The humidifier includes a tank for storing a volume of water, wherein the tank has an inflow port for receiving a gas stream, and an outflow port for allowing humidified gas at a required SH to exit the tank. The temperature of the water and/or the pressure of the supplied gas are controlled for the required SH.

According to an aspect is provided a method for sterilizing a medical instrument. The method includes placing the medical instrument in a chamber and supplying a humidified gas stream to the medical instrument. The humidified gas stream has a controlled specific humidity. The gas stream having the controlled specific humidity is obtained by providing a gas stream into a tank storing a volume of water, controlling a temperature of the water and/or a pressure of the supplied gas for the required specific humidity, and flowing the humidified gas stream having the controlled specific humidity from the tank.

More in general is provided a method for controlling specific humidity of a gas stream. The method includes providing a gas stream into a tank storing a volume of water. The method includes controlling a temperature of the water and/or a pressure of the supplied gas for the required specific humidity. The method includes flowing the humidified gas stream having the controlled specific humidity from the tank.

It will be appreciated that any of the aspects, features and options described in view of the apparatus apply equally to the device and method, and vice versa. It will also be clear that any one or more of the above aspects, features and options can be combined.

### BRIEF DESCRIPTION OF THE DRAWINGS

Embodiments of the present invention will now be described in detail with reference to the accompanying drawings in which:

FIG. 1 shows a schematic representation of an example of an apparatus for sterilizing a medical instrument;

FIG. 2 shows a schematic representation of an example of a humidifier;

FIG. 3 shows a schematic representation of a method;

FIG. 4 shows a schematic representation of a method;

FIG. 5 shows an example of pressure curves;

FIG. 6 shows a schematic representation of an example of an apparatus for sterilizing a medical instrument; and

FIG. 7 shows a schematic representation of an example of an apparatus for sterilizing a medical instrument.

### DETAILED DESCRIPTION

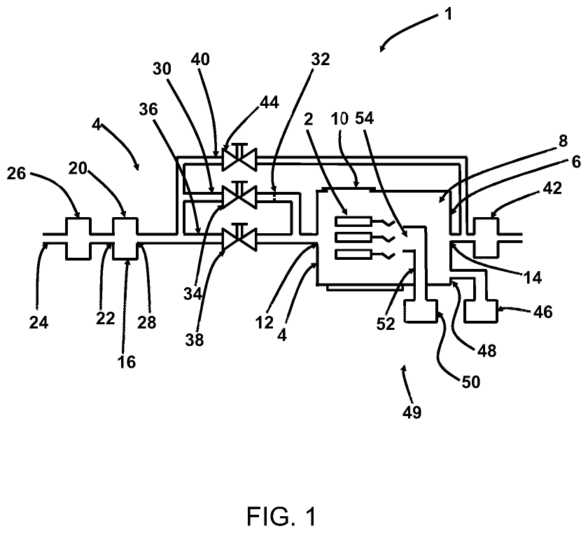

FIG. 1 shows an example of a schematic view of a sterilization apparatus 1 for sterilizing medical instruments, such as dental instruments 2. The apparatus 1 includes a sterilizing chamber 4. The sterilizing chamber 4 is arranged for placing the medical instrument 2 to be sterilized therein. In this example, the chamber 4 is arranged for placing a plurality of medical instruments 2 to be sterilized therein. Here, the medical instruments 2 are dental instruments. The chamber 4 includes walls 6 forming an internal space 8 for receiving the medical instruments 2. In this example, the chamber 4 has a door 10 for allowing the medical instruments 2 to be inserted into and extracted from the internal space 8 of the chamber 4. The chamber 4 includes a sterilizing agent supply port 12. The chamber 4 includes an exhaust port 14.

The apparatus 1 includes a sterilizing agent source 16. The sterilizing agent source 16 is arranged for providing a sterilizing agent. In this example, the sterilizing agent includes at least partially recombined ionized humidified air. In this example, the sterilizing agent source 16 includes a plasma source 20. The plasma source 20 includes an output port 28 in communication with the sterilizing agent supply port 12 of the chamber 4.

A first duct 30 extends between the plasma source 20 output port 28 and the sterilizing agent supply port 12 of the chamber 4. The first duct 30 includes a choke 32. In this example, the first duct 30 further includes a first valve 34. The first valve 34 is arranged for selectively opening and closing the first duct 30. Here the first valve 34 is positioned upstream of the choke 32. It will be clear that alternatively the first valve 34 may be positioned downstream of the choke 32.

The choke 32 is arranged for causing choked flow of the sterilizing agent from the plasma source 20 to the chamber 4 when the chamber is at the reduced pressure. The choked flow is a fluid dynamic condition associated with the Venturi effect. When a gas stream at a given pressure and temperature passes through a choke, also referred to as restriction or constriction, into a lower pressure environment the gas velocity increases. At initially subsonic upstream conditions, the conservation of mass principle requires the gas velocity to increase as it flows through the smaller cross-sectional area of the choke. At the same time, the Venturi effect causes the static pressure, and therefore the density, to decrease at the choke. Choked flow is a limiting condition where the mass flow will not increase with a further decrease in the downstream pressure for a fixed upstream pressure and temperature. The physical point at which the choking occurs for adiabatic conditions is when the velocity at the exit of the choke is at sonic conditions; i.e., at a Mach number of 1. Hence, thanks to the choke 32 the pressure in the plasma source 20 does not decrease below a predetermined threshold pressure when the pressure in the chamber 4 is reduced further below such predetermined threshold pressure.

Steady-state choked flow occurs when the pressure downstream of the choke falls below a critical value relative to the pressure upstream of the choke. The critical pressure value p* can be calculated from the following equation.

$$\frac{{p}^{*}}{{p}_{0}} = {( \frac{2}{γ + 1} )}^{\frac{γ}{γ - 1}} \qquad EQ 2$$

Herein p₀ is the absolute upstream pressure and γ the heat capacity ratio cₚ/cᵥ of the gas or gas mixture. For air the heat capacity ratio γ is about 1.4. Thus for air p*=0.528p₀.

When the gas velocity is choked, the equation for the mass flow rate is as follows.

$$\dot{m} = {C}_{d} A \sqrt{γ {ρ}_{0} {{p}_{0} ( \frac{2}{γ + 1} )}^{\frac{γ + 1}{γ - 1}}} \qquad EQ 3$$

Herein \dot{m} is the mass flow rate in kg/m2, C_{d} the discharge coefficient (dimensionless), A the cross-sectional area of the hole in m², ρ₀ the real gas density at total pressure p₀ and temperature T₀ in kg/m³, and T₀ the absolute upstream temperature of the gas in K. The discharge coefficient C_{d} is the ratio of the actual discharge to the theoretical discharge, i.e. the ratio of the mass flow rate at the discharge end of the nozzle to that of an ideal nozzle which expands an identical working fluid from the same initial conditions to the same exit pressures. The discharge coefficient C_{d} is generally in the range of 0.5-1. The Discharge coefficient can e.g. be on the order of 0.6 (e.g. sharp edged orifice) to 0.8 (e.g. longer hole). The discharge coefficient for a specific choke can easily be determined by comparing a measured mass flow rate to the theoretical mass flow rate (C_{d}=1).

Hence, by properly designing the flow restriction properties of the choke 32 it is possible to prevent the pressure in the plasma source 20 to decrease below the threshold pressure, while the sterilizing chamber 4 is evacuated. Thus, the plasma source 20 can be operated continuously at nearly constant pressure, e.g. at near-ambient pressure. Hence, composition of the at least partially recombined gas mixture from the plasma source 20 can remain constant, or at least nearly constant, i.e. sufficiently constant, despite reducing the pressure in the sterilizing chamber 4.

In the example of FIG. 1 a second duct 36 extends between the plasma output port 28 and the sterilizing agent supply port 12 of the chamber 4. The second duct 36 includes a second valve 38. The second valve 38 is arranged for selectively opening and closing the second duct 36.

In the example of FIG. 1 a third duct 40 extends between the plasma output port 28 and a destructor 42. The destructor 42 is arranged for destructing airborne components. In this example, the third duct 40 further includes a third valve 44. The third valve 44 is arranged for selectively opening and closing the third duct 40. In FIG. 1 the destructor 42 is further connected to the exhaust port 14 of the chamber 4 for destructing any contaminants carried by the exhausted sterilizing agent.

In this example, a pump 46 is connected to a pumping port 48 of the chamber 4. The apparatus 1 in this example includes a temperature control unit 49. In this example, the temperature control unit 49 includes a cooling unit 50. The cooling unit 50 is arranged for cooling the medical instruments 2. In FIG. 1, the cooling unit 50 is arranged for cooling the instruments 2 prior to placing the instruments 2 in the chamber 4. The cooling unit 50 in this example includes a gas conduit 52 for cooling the medical instrument using a gas, here air. The gas conduit 52 includes a mouth 54, here nozzles, pointing a stream of the gas onto the medical instrument 2.

The plasma source 20 includes an input port 22 for feeding a humidified air stream into the plasma source 20. In FIG. 1 the input port 22 is connected to an air stream supply 24 via a humidifier 26. In this example, the humidifier 26 is a device for controlling specific humidity, SH, of a flow of a gas therethrough. It will be appreciated that the gas can be a gas mixture. It will be appreciated that the gas, or gas mixture, can include a vapor.

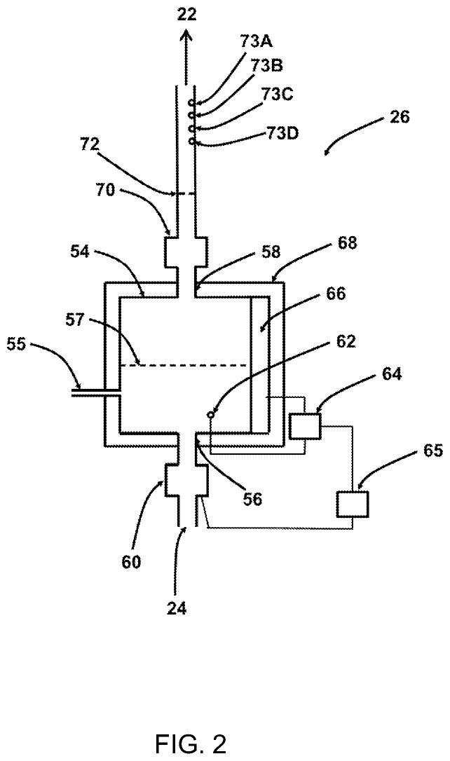

FIG. 2 shows a schematic depiction of a more detailed example of a humidifier. The humidifier 26 includes a tank 54. In this example the tank 54 is partially filled with water. The tank has an inflow port 56 for receiving the gas, here from the air stream supply 24. The air stream supply 24 can e.g. be a compressed air supply, e.g. at approximately 6 bar(a). Here the inflow port 56 is arranged to have the gas enter the volume of water below the water surface 57. The tank 54 has an outflow port 58 for allowing humidified gas at the required SH to exit the tank 54. Here, the outflow port 58 is in communication with a gas head inside the tank 54. Here, the apparatus includes a water supply port 55 for maintaining the water level within a predetermined interval. The water supply port 55 can be provided with an automatic filling device, e.g. a float operated valve or a liquid level operated valve for automatically maintaining the water level with a predetermined interval.

By flowing, e.g. bubbling, the gas through the water, the relative humidity, RH, of the gas will be at, or near, 100%. Also, by flowing the gas through the water, the temperature of the gas will be equal to, or close to, the temperature of the water. It will be appreciated that a person of skill in the art can easily determine, e.g. on the basis of some simple experiments, a volume of liquid required for ensuring that in the gas head of the tank the relative humidity, RH, of the gas will be at 100%, and that the temperature of the gas will be equal to the temperature of the water.

In the example of FIG. 2 the humidifier 26 includes a pressure regulator 60 in communication with the inflow port 56. Here the pressure regulator 60 is a control valve that reduces the input pressure of a gas to a desired value at its output. Here the pressure regulator 60 is adjustable, such that a pressure at the output of the regulator can be adjusted to a desired value. Thus the pressure in the tank 54 can be adjusted to a desired value.

In the example of FIG. 2 the humidifier 26 is arranged for controlling the temperature of the water in the tank 54. Here, the humidifier 26 includes a temperature sensor 62, a temperature controller 64, and a heater 66. A thermal insulation 68 may be provided around the tank 54 for conserving energy and for minimizing temperature fluctuations inside the tank 54. The temperature controller 64 is arranged for controlling heat of the heater 66 for maintaining the water at a controlled temperature on the basis of a temperature measured by the temperature sensor 62. Here the temperature at which the controller 64 maintains the water is adjustable, such that the temperature of the water can be adjusted to a desired value. Thus the temperature of the water in the tank 54 can be adjusted to a desired value.

The specific humidity, SH, of the gas in the gas head in the tank 54 is dependent on the relative humidity, RH, of the gas, the pressure and the temperature. As described above, the RH can be set to 100% in the tank. The temperature of the gas can be set by setting the temperature of the water in the tank, e.g. using the temperature controller 64. The pressure of the gas in the gas head can be set by setting the feed pressure using the pressure regulator 60. For example, for a desired specific humidity of 10 grams of water vapor per kilogram of gas, and a water temperature of 40° C., using equation EQ1 the required pressure can be calculated to be Pₜₒₜ(40, 10)=4.623 bar(a). Thus, by setting the pressure regulator 60 to 4.623 bar(a) and setting the temperature controller 64 to 40° C., gas flow having a specific humidity of 10 g/kg can be obtained.

In the example of FIG. 2 the humidifier 26 further includes a processor 65. The processor 65 is arranged for receiving or determining a desired SH value and for determining values for a required temperature and/or required pressure on the basis of the desired SH value. The processor can e.g. determine a combination of suitable required temperature and required pressure on the basis of equation EQ1 as set out hereinabove. The processor can be arranged to simultaneously control both the temperature and the pressure and maintain both at the required value. It is also possible that the processor monitors the pressure, in real-time (or at least with minimum delay time) determines the required temperature for obtaining the desired SH at the measured pressure, and control the temperature to be at the required temperature in real-time. It is also possible that the processor monitors the temperature, in real-time (or at least with minimum delay time) determines the required pressure for obtaining the desired SH at the measured temperature, and control the pressure to be at the required pressure in real-time.

In this example, the processor is arranged for receiving an indication representative of a desired sterilization parameter. The desired sterilization parameter can e.g. be a desired reactive component content of the plasma. The indication representative of the desired sterilization parameter can e.g. be one of a set of predetermined settings of the sterilization apparatus. The predetermined settings can e.g. relate to different instruments to be sterilized, different grades of contamination of the instruments to be sterilized, different levels of sterilization, or the like. The processor in this example determines the desired SH value on the basis of the indication representative of the desired sterilization parameter. The processor can e.g. look up the desired SH value corresponding to the desired sterilization parameter in a database or memory. In the example of FIG. 2 a further pressure regulator 70 is provided at the outflow port 58 of the humidifier 26. Here, the further pressure regulator is set to a pressure that is lower than the pressure inside the tank 54. Thus, the humidified gas can be expelled from the humidifier 26 at a regulated pressure that is independent from the gas pressure inside the tank. Hence, feed pressure towards the plasma source and pressure selected for setting the desired SH can be chosen, and controlled, independently. Further, in the example of FIG. 2 a second choke 72 is in communication between the outflow port 58 of the humidifier 26 and the input port 22 of the plasma source 20. This second choke 72 can be used for further reducing the pressure of the humidified gas fed towards the plasma source 20. Further, the second choke 72 can be dimensioned to define a mass flow of humidified gas to the plasma source. It is noted that when reducing the pressure and/or temperature of the gas stream, such as downstream of the tank 54, the SH of the gas stream does not change. The RH of the gas stream, however, does change by said reducing of the pressure and/or temperature. Therefore, piping connecting the tank to the plasma source may be thermally insulated for preventing saturation, or even condensation, of the gas stream to occur upstream of, or in, the plasma source 20.

In this example, a connection from the outflow port 58 of the humidifier 26 to the input port 22 of the plasma source 20 includes one or more of a RH sensor 73A, a temperature sensor 73B, a pressure sensor 73C, and a flow sensor 73D. These sensors can be used for verifying a mass flow towards the plasma source and/or for verifying the SH of the gas flowing to the plasma source 20. The SH can be calculated from the measured RH, temperature and pressure, on the basis of equation EQ1.

In this example, the SH value determined from the readings of the sensors 73A, 73B, 73C is used for verification only. In this example, there is no data connection between the sensors 73A, 73B, 73C and the processor 65 or controller 64. It will be appreciated that it is also possible to use the determined SH value for adjusting the pressure and or temperature in the tank 54 for controlling the SH.

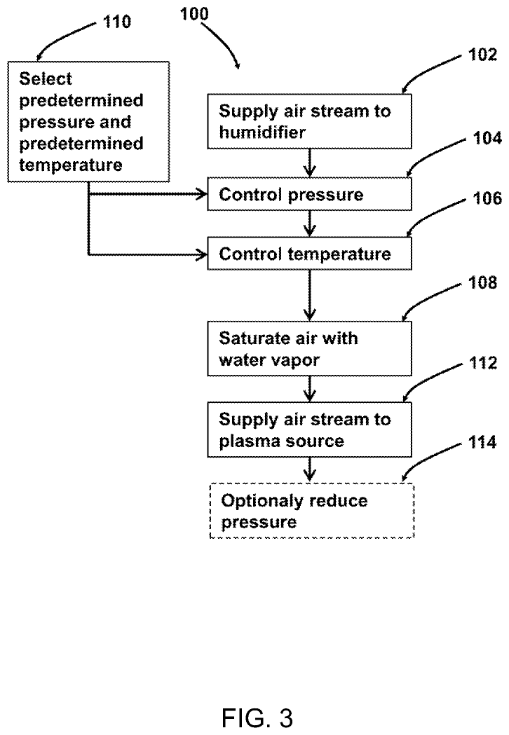

The apparatus as described in relation to FIGS. 1 and 2 can be used in the following exemplary first method 100, see FIG. 3. An air stream is supplied 102 from the air stream supply 24 via the pressure regulator 60 to the inflow port 56 of the humidifier 26 for setting the specific humidity of the air stream to a predetermined SH value. The pressure of the air stream is controlled 104 by the pressure regulator 56 to a predetermined pressure value. The air stream enters the tank 54 below water level. The water is controlled to be at a predetermined temperature value by temperature controller 64. Thereby the temperature of the air in the tank 54 is controlled 106 to the predetermined temperature value. Also, the air is saturated 108 with water vapor, i.e. RH becomes 100% at the predetermined pressure and predetermined temperature. Depending on the humidity of the air supplied to the humidifier 26, the humidifier 26 can add or remove water from the air such that at the exit of the humidifier 26 an air stream with a predetermined SH is obtained. The predetermined pressure and predetermined temperature had been chosen 110 such that, at saturation, the specific humidity of the gas corresponds to the predetermined SH value.

The gas having the predetermined SH value is fed 112 from the outflow port 58 of the humidifier 26, towards the plasma source 20. Here, the pressure of the gas stream is reduced 114 by the further pressure regulator 70 and the second choke 72. The air stream entering the plasma source 20 has the predetermined SH value. The specific humidity of the air entering the plasma source 20 can e.g. be 10±1 g/kg (grams of water per kg of air).

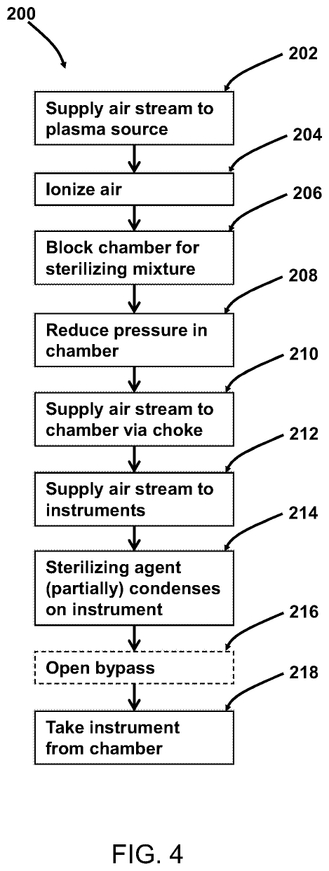

According to a second method 200, see FIG. 4, the gas having the predetermined SH value is fed 202 from the outflow port 58 of the humidifier 26, to the plasma source 20. In the plasma source 20 the air is at least partially ionized 204. The ionized air is fed to the output port 28. With the first and second valves 34, 38 closed, preventing 206 the sterilizing mixture to enter the chamber 4, the sterilizing mixture may be fed to the destructor 42 via the third duct 40 with the third valve 44 opened. Hence, the plasma source 20 can be operated continuously. The sterilizing mixture can be fed to the chamber 4 when needed, and fed to the destructor 42 when not needed in the chamber 4. Thus, the plasma source 20 is immediately ready for providing the sterilizing mixture 18 to the chamber 4 when required.

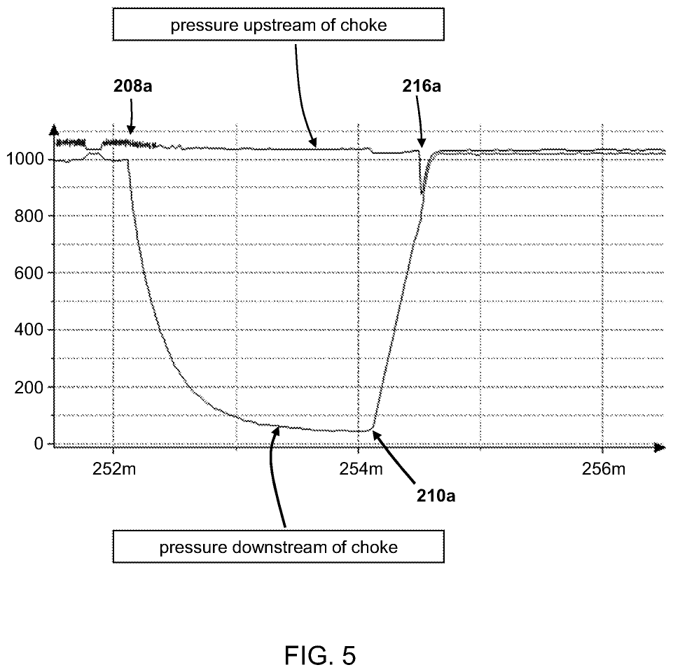

The medical instruments 2 to be sterilized are placed inside the chamber 4. Then, the pressure in the chamber 4 is reduced 208 by the pump 36. In this example the pressure is reduced to approximately 50 mbar FIG. 5 shows a graph of the pressure as a function of time upstream of the choke 32, here the pressure in the plasma source 20, (upper curve) and the pressure downstream of the choke 32, here in the sterilization chamber 4 (lower curve). FIG. 5 shows that the pressure in the chamber 4 starts to decrease from the moment 208a the pump is started. It is noted that the pressure upstream of the choke 32 does not decrease yet as the first and second valves 30, 36 are still closed in this example. This removes a large portion of the gasses that entered the chamber 4 while placing the medical instruments 2 therein. Next, the first valve 34 is opened at moment 210a. Hence, the sterilizing mixture is fed 210 into the chamber 4 via the choke 32. Thus, the pressure in the chamber 4 will be raised by feeding the sterilizing mixture into the chamber 4 as can be seen in FIG. 4. The choke 32 in this example is dimensioned such that the pressure upstream of the choke 32 is maintained at approximately 1 bar. Hence, the pressure in the plasma source 20 can in this example not drop below a predetermined threshold pressure of approximately 1 bar. Here the flow through the choke is limited to about 10 liters per minute at 1 bar. Thereto, in this example, the choke has a round aperture of 0.785 mm.

In this example, with the first valve opened, the flow of the ionized humidified air from the plasma source 20, at least partly, recombines while flowing into the chamber 4. The sterilizing agent formed by the at least partly recombined ionized humidified air then contacts 212 the medical instruments 2 to be sterilized. Optionally, if the medical instruments 2 are, or have been, cooled, the sterilizing agent, at least partially, condenses 214 onto the medical instruments 2 and sterilizes the medical instruments 2. If the walls 6 of the chamber 4 are not cooled, less cooled than the medical instruments, or even heated, condensation of the sterilizing agent onto the walls 6 can be prevented.

In this example, as an option, the second valve 38 is opened 216 at moment 216a when the pressure in the chamber 4 rises to about 850 mbar, i.e. when the pressure in the chamber exceeds a predetermined threshold opening pressure of, here, 850 mbar. The opening of the second valve causes sterilizing agent to rush into the chamber 4 via the second duct 36, i.e. bypassing the choke 32. When the second valve 38 is open, the first valve 34 may be closed if desired. Hence, when the pressure in the chamber 4 is not too low, i.e. here not below the predetermined threshold opening pressure of in this example 850 mbar, the second valve 38 can be opened to allow a greater mass flow of sterilizing mixture than is achievable through the choke 32. As the pressure in the chamber 34 is above the threshold opening pressure at that time, the pressure in the plasma source cannot drop below the threshold opening pressure.

The medical instruments 2 may be taken from the chamber 218 immediately or may remain in the chamber 4 for some time for additional exposure to the sterilizing agent. The sterilizing agent may be fed to the destructor 42 via the exhaust port 14.

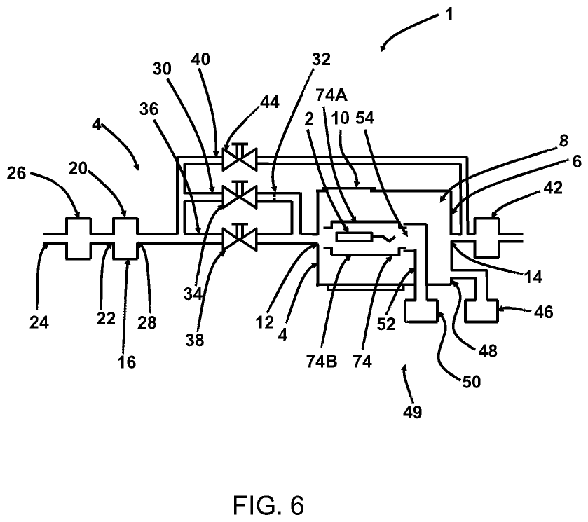

FIG. 6 shows a schematic representation of an example of an apparatus 1 for sterilizing a medical instrument 2. The example of FIG. 6 is similar to the example of FIG. 1. A main difference is that the apparatus 1 of FIG. 6 further includes a container 74. The container 74 is arranged for holding the medical instrument 2, here for holding a plurality of medical instruments 2. The container in this example includes a tray 74A and a lid 74B. The container 74 can be opened by removing the lid 74B from the tray 74A for placing one or more medical instruments 2 inside the container 74. The container 74 is arranged for being placed in the chamber 4. The chamber 4 can include guides for holding the container 74. The apparatus 1 in this example is arranged for opening the container inside the chamber 4. In the example of FIG. 6 the cooling unit 10 is arranged for cooling the container 74 to below the temperature of the chamber 4. Hence, the medical instruments 2 in the container 74 can easily be cooled together with the container 74. Also, hence the container 74, which can be contaminated as well, can easily be sterilized.

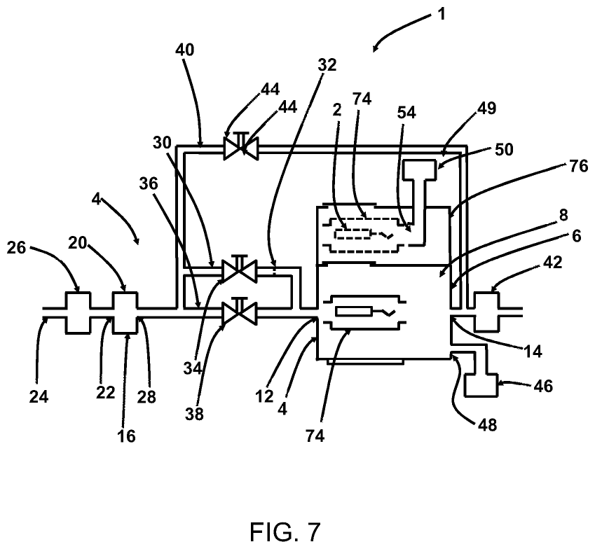

FIG. 7 shows a schematic representation of an example of an apparatus 1 for sterilizing a medical instrument 2. The example of FIG. 7 is similar to the example of FIG. 6. A main difference is that the apparatus 1 of FIG. 7 further includes a cooling chamber 76. The cooling chamber 76 is arranged for holding the medical instrument 2, here for holding the container 74 holding medical instruments 2 while cooling the medical instrument(s) 2 and optionally the container 74. In this example the medical instruments 2, here in the container 74, are cooled in the cooling chamber 58 and then transferred to the chamber 4 for sterilization. The apparatus 1 can include a handler unit for transferring the medical instruments 2 and/or the container 74 from the cooling chamber 76 to the sterilization chamber 4 after cooling.

Herein, the invention is described with reference to specific examples of embodiments of the invention. It will, however, be evident that various modifications and changes may be made therein, without departing from the essence of the invention. For the purpose of clarity and a concise description features are described herein as part of the same or separate embodiments, however, alternative embodiments having combinations of all or some of the features described in these separate embodiments are also envisaged.

In the example of FIG. 1 the gas stream having the predetermined SH value is fed from the humidifier to the plasma source. It is also possible that the gas stream having the predetermined SH value is fed from the humidifier to the chamber without passing a plasma source.

It will be appreciated that the humidifier as described herein can also be used in alternative sterilizing apparatus than described in view of FIGS. 1, 6 and 7.

In the example of FIG. 1 the cooling unit is arranged for cooling the medical instrument in the chamber. It is also possible that alternatively, or additionally, the cooling unit is arranged for cooling the medical instrument prior to placing the medical instrument inside the chamber, e.g. as disclosed in view of FIG. 7.

It is possible that the apparatus further includes a washing unit arranged for washing and/or rinsing the medical instruments prior to sterilization. Preferably, the medical instruments are dried prior to sterilization. The cooling gas stream, optionally including the atomized water, can be supplied to the washed medical instruments for drying and cooling the medical instruments.

However, other modifications, variations, and alternatives are also possible. The specifications, drawings and examples are, accordingly, to be regarded in an illustrative sense rather than in a restrictive sense.

For the purpose of clarity and a concise description features are described herein as part of the same or separate embodiments, however, it will be appreciated that the scope of the invention may include embodiments having combinations of all or some of the features described.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage.

1. A sterilization apparatus for sterilizing medical instruments, including:
a device for controlling specific humidity, SH, of flow of a gas or gas mixture, including a tank for storing a volume of water,
wherein the tank has an inflow port for receiving the gas or gas mixture;
wherein a temperature of the water and/or a pressure of the received gas or gas mixture are controlled for a required SH; and
wherein the tank has an outflow port for allowing humidified gas or gas mixture at the required SH to exit the tank.

2. The apparatus of claim 1, wherein the inflow port is arranged to have the gas or gas mixture enter the volume of water below a water surface thereof.

3. The apparatus of claim 1, wherein the outflow port is in communication with a gas head inside the tank.

4. The apparatus of claim 1, including a processor arranged for retrieving a desired SH value and for determining values for a required temperature and/or required pressure on the basis of the desired SH value.

5. The apparatus of claim 1, including a temperature sensor, and temperature controller for maintaining the water at a controlled temperature.

6. The apparatus of claim 4, wherein the processor is arranged for receiving a controlled temperature value as input, and for determining the required pressure on the basis of the desired SH and the controlled temperature value.

7. The apparatus of claim 1, including a pressure regulator for maintaining a pressure in the tank at a controlled pressure.

8. The apparatus of claim 4, wherein the processor is arranged for receiving a controlled pressure value as input, and for determining the required temperature on the basis of the desired SH and the controlled pressure value.

9. The apparatus of claim 4, wherein the processor is arranged for controlling the temperature and/or pressure in the tank on the basis of the required temperature and/or required pressure.

10. The apparatus of claim 4, wherein the processor is arranged for receiving an indication representative of desired sterilization parameter and determining the desired SH value on the basis thereof.

11. The apparatus of claim 1, including a water supply port for maintaining a water level of the volume of water within a predetermined interval.

12. The apparatus of claim 1, including a plasma source having an ionization chamber in communication with the device for controlling specific humidity.

13. The apparatus of claim 12, wherein the outflow port of the device is connected to a gas input of the ionization chamber via a choke.

14. The apparatus of claim 12, including one or more of: a RH sensor, temperature sensor, pressure sensor, and flow sensor, in a connection from the outflow port of the device to a gas input of the ionization chamber.

15. The apparatus according to claim 1, including:
a chamber arranged for placing a medical instrument therein; and
a temperature control unit arranged for controlling a temperature of the medical instrument and/or the chamber such that the temperature of the medical instrument is below the temperature of chamber for allowing a sterilizing agent to at least partially condense onto the medical instrument.

16. A method for sterilizing a medical instrument, including:
placing the medical instrument in a chamber;
supplying a humidified gas stream to the medical instrument, wherein the humidified gas stream has a controlled specific humidity and is obtained by:
providing a gas stream into a tank storing a volume of water,
controlling a temperature of the water and/or a gas pressure of the supplied humidified gas stream for a required specific humidity,
flowing the humidified gas stream having the controlled specific humidity from the tank.

17. The method of claim 16, including having the humidified gas stream enter the tank below a water surface of the volume of water.

18. The method of claim 16, including flowing the humidified gas stream having the controlled specific humidity from a gas head inside the tank.

19. The method of claim 16, including using a processor for retrieving a desired SH value and for determining values for a required temperature and/or required pressure on the basis of the desired SH value.

20. The method of claim 16, including using a temperature sensor, and using temperature controller for maintaining the water at the controlled temperature.

21. The method of claim 19, including the processor receiving a controlled temperature value as input, and determining a required pressure on the basis of the desired SH and the controlled temperature value.

22. The method of claim 16, including controlling the gas pressure using a pressure regulator for maintaining a pressure in the tank at a controlled pressure.

23. The method of claim 19, including the processor receiving a controlled pressure value as input, and determining a required temperature on the basis of the desired SH and the controlled pressure value.

24. The method of claim 19, including the processor controlling the temperature and/or a pressure in the tank on the basis of the required temperature and/or required pressure.

25. The method of claim 19, including the processor receiving an indication representative of desired sterilization parameter and determining the desired SH value on the basis thereof.

26. The method of claim 16, including automatically maintaining a water level of the volume of water in the tank within a predetermined interval.

27. The method of claim 16, including at least partly ionizing the humidified gas stream having the controlled specific humidity using a plasma source prior to supplying the humidified gas stream to the medical instrument.

28. The method of claim 27, including flowing the humidified gas stream having the controlled specific humidity from the tank to the plasma source via a choke.

29. The method of claim 27, including measuring one or more of: relative humidity, temperature, pressure, and flow, in a connection from the tank to the plasma source.

30. The method according to claim 16, including controlling a temperature of the medical instrument and/or the chamber such that the temperature of the medical instrument is below the temperature of chamber for allowing a sterilizing agent to at least partially condense onto the medical instrument.
